(19) United States
(12) Patent Application Publication
(10) Pub. No.: US 2006/0041191 A1
(43) Pub. Date: Feb. 23, 2006

(54) Universal endoscope

(76) Inventors: Sergey Popovich, Krasnoyarsk (RU); Andrey Chursin, Krasnoyarsk (RU)

Correspondence Address: Chak Leung, 1242 Byron Street, Palo Alto, CA, 94301, US

(21) Appl. No.: 10/922,304

(22) Filed: Aug. 20, 2004

(51) Int. Cl. A61B 1/00

(52) U.S. Cl. 600/175; 600/129

(57) ABSTRACT

A universal endoscope of the invention comprises an outer shell with quickly and conveniently replaceable inner parts of the endoscope, such as a smooth end tip assembly which is inserted into the outer shell for filling the interior of the outer shell and for guiding the endoscope during insertion into the patient's lumen, e.g., rectum. For this purpose the end of the end tip that projects from the distal part of the outer shell has a smooth tip that merges the external shape of the aforementioned distal part. Upon completion of the endoscope insertion, the end tip assembly is removed for opening the interior of the outer shell for providing access of various replaceable instruments and tools to the area of the interests. This may be biopsy samplers, polyp cutters, etc. Reliable fixation of the end tip assembly and tools is provided due to the use of a metal-plastic friction pair between the inner taper on the lock member of the outer shell and the outer taper on the inserted member, while quick and convenient connection and disconnection is achieved due to provision of a lead threads on the aforementioned lock member and the inserted member.

### FIELD OF THE INVENTION

The invention relates to medical instrumentation, in particular, to universal endoscopes, and more specifically, to rectoscopes for performing intracavitory examination of the strait intestine and sigmoid section of the anal canal and for performing surgical operations in the observed zone of interest.

### BACKGROUND OF THE INVENTION

The colon is 5 ft (1.5 m) to 6 ft (1.8 m) long. Sigmoidoscopy is a thorough examination of the last 2 ft (0.6 m) of the lower colon (sigmoid colon). Sigmoidoscopy is most commonly used to screen for colon cancer. About half of all colon tumors and polyps can be found using sigmoidoscopy. These examinations are done using different viewing instruments that are inserted into the anus, rectum, or colon. Depending on specific application, the aforementioned viewing instruments are known as anoscopes, endoscopes, proctoscopes, sigmoidoscopes, etc. Since the principle of the present invention is applicable to aforementioned instruments of any type, in the context of the present invention the instrument will be called as “endoscope” which is the most general term for the instruments of the aforementioned type. Although such endoscopes are typically lighted tube that may be rigid or flexible, the invention will be described as an example of its application with a reference to a rigid sigmoidoscope, which is a tubular instrument of about 10 in. (25.4 cm) to 12 in. (30.5 cm) long and 1 in. (2.5 cm) in diameter. It allows the health professional to look into the rectum and a portion of the lower large intestine (colon). It is understood that the field of application of the invention is not limited to a sigmoidoscope.

There exist a great variety of endscopes, in particular rectoscopes, simoidoscopes, etc. of different configurations and designs of flexible and rigid types. As has been mentioned above, these instruments, especially of a rigid type, are common in that they comprise a rigid outer shell with various replaceable optical means for observation and with tools for surgical procedures such as a removal of polyps, taking biopsy samples, etc. Roughly, from the structural point of view, the aforementioned endoscopes can be divided into two groups: 1) specialized endoscopes having a construction that depends on the structure and requirements of specific tools associated with the purposes of the endoscopes; and 2) universal endoscopes having a construction that does not depend on the specific tools and that allows the use of any tools required by the procedure.

An example of the specialized endoscopes is the one disclosed, e.g., in U.S. Pat. No. 6,592,508 issued in 2003 to S. Ravins, et al. that describes a fiber-optic guided intrerstitial manual applicator and seed cartridge. The apparatus includes an implantation needle having a bore extending longitudinally therethrough from a proximal end to a distal end of the needle, the needle bore being adapted to permit at least one seed to pass therethrough. An elongated plunger extends longitudinally through the implantation device in aligned relation to the needle bore and is selectively movable in the longitudinal direction relative the needle from a retracted position spaced apart from the needle to an extended position wherein the plunger is advanced through the needle bore to eject at least one of the seeds through the bore, out of the distal end of needle and into the target area. An optical device is carried by and operatively connected to the plunger to provide visual assistance to an operator of the implantation device to guide and verify implantation of the ejected seed into the target area. A seed cartridge is releasably connected to the implantation device to store unused seeds and to load the seeds into the implantation device.

A disadvantage of the above-described device is that it has a complicated construction which is inconvenient for replacement of the tools and for sterilization, since replacement of the tools requires that the outer shell be removed from the patient's body cavity and disconnected from the housing of the instruments. Sterilization is complicated because of the intricate shape of the components.

Another example of specialized endoscopes of the aforementioned first group is a rectoscope disclosed in U.S. Pat. No. 6,458,077 issued in 2002 to M. Boebel. The rectoscope has a working insert which may be inserted into a coupling housing sitting on the proximal side on an instrument shank and is releasably connectable to said housing and which includes a seal, carried by a carrier, which has at least one elastic socket for the sealed leading-through of auxiliary instruments and which seals a first axial passage formed in the carrier with respect to the coupling housing. The seal consists of a base body which is located in a holder and which with each socket forms a one-piece component of elastic material, and the holder includes an arrangement for the releasable connection to the carrier. Since this instrument is provided with means for supply of gas in order to expand the body lumen into which the instrument is inserted, it requires the use of a sealing device. This makes the construction of the instrument complicated and inconvenient for sterilization.

U.S. Pat. No. 6,039,687 issued in 2000 to Karl Storz describes a rectoscope for examination of the rectum that can be considered as an endoscope of the second, i.e., universal group. The rectoscope, which is especially suited for the examination of the rectum, is provided with a tubular outer shell that has a lighting device attached sidewise to the proximal end of the outer shell and an optical viewing device that is attached to the proximal end face of the shell through a detachable coupling. The internal side of the outer shell is designed semi-reflecting so that the internal side of the tube serves as a light transmitter for the light emerging from the light exit area. In the preferred embodiment, a flange is provided which covers the proximal opening of the outer shell like a kind of cover and which is detachably connected to the tube by means of the aforementioned coupling. Furthermore, the flange is provided with a channel or opening for insertion of a tool. This opening is offset with respect to the central axis of the shell.

A disadvantage of the aforementioned device is that for use of tools other than those that could be inserted into the small-diameter offset opening, the flange has to be disconnected from the coupling and removed together with the viewing device, if the latter is attached thereto, for opening the proximal end of the endoscope. The connection parts are made of metal and have cavities, which are difficult for sterilization. The viewing device provides only a distant observation, i.e., from the proximal end of the instrument and not in a close proximity from the area of the interest.

### SUMMARY AND OBJECTS OF THE INVENTION

It is an object of the invention to provide a universal endoscope for proctological purposes, which is simple in construction, convenient for use and sterilization, can be provided with an optical observation unit located on the distal end of the outer shell in direct proximity to the area of interest, provides quick one-step removal of the observation and access to the area of interest for any tool that could be inserted into the outer shell of the endoscope through the completely open proximal end, has an improved locking device for the replaceable optics with the use of a metal-plastic friction pair that improves sliding and prevents jamming of connection components.

A universal endoscope of the invention comprises an outer shell with quickly and conveniently replaceable inner parts of the endoscope, such as a smooth end tip assembly which is inserted into the outer shell for filling the interior of the outer shell and for guiding the endoscope during insertion into the patient's lumen, e.g., rectum. For this purpose the end of the end tip that projects from the distal part of the outer shell has a smooth tip that merges the external shape of the aforementioned distal part. Upon completion of the endoscope insertion, the end tip assembly is removed for opening the interior of the outer shell for providing access of various replaceable instruments and tools to the area of the interests. This may be biopsy samplers, polyp cutters, etc. Reliable fixation of the end tip assembly and tools is provided due to the use of a metal-plastic friction pair between the inner taper surface on the lock member of the outer shell and the outer taper surface on the inserted member, while quick and convenient connection and disconnection is achieved due to provision of a lead threads on the aforementioned lock member and the inserted member.

### BRIEF DESCRIPTION OF THE DRAWINGS

FIG. 1 is a longitudinal view of the endoscope with one half of it being shown in a longitudinal section.

FIG. 2 is fragment A of FIG. 1 shown on a larger scale.

FIG. 3 is a longitudinal partially sectional view of an end tip assembly shown in FIGS. 1 and 2.

FIG. 4 is a longitudinal partially sectional view of a lock member used in the endoscope of the invention for locking instruments and tools in the outer shell of the endoscope.

FIG. 5 is a longitudinal partially sectional view of an optical viewing device as an example of a replaceable unit that can be connected to the proximal end of the endoscope.

FIG. 6 is a longitudinal partially sectional view of an adapter screw member for use as an adapter that allows connection of various tools and instruments for access to the zone of observation or operation.

### DETAILED DESRIPTION OF THE INVENTION

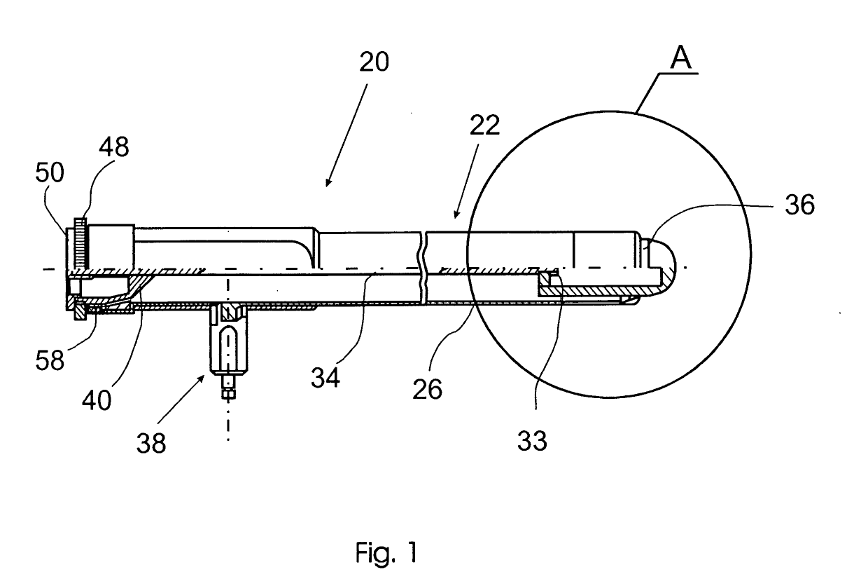

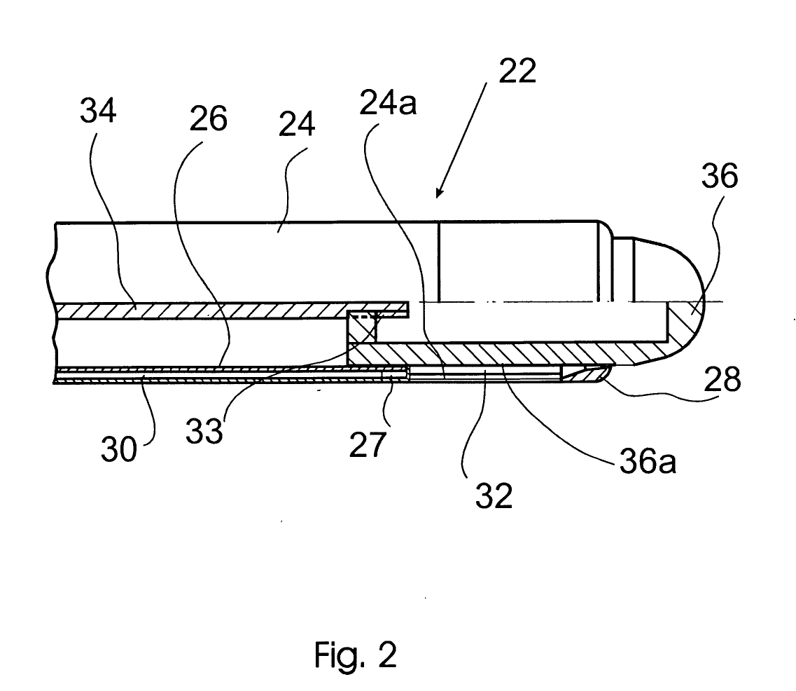

The endoscope of the invention, which in general is designated by reference numeral 20, is shown in FIG. 1 which is a longitudinal view of the endoscope with one half of it being shown in a longitudinal section. The endoscope 20 has an outer shell 22 intended for inserting into the patient's rectum and for installation of surgical or observation tools. As shown in FIG. 2, which is fragment A of FIG. 1 shown on a larger scale, the outer shell 22 consists of an outer tube 24 and an inner sleeve 26 with an annular space 27 between them and with a cylindrically-shaped optical fiber bundle 30 of a lighting device which is inserted into the aforementioned annular space 27.

The outer and inner tubes 24 and 26 are made from a medically acceptable metal such as stainless steel or a biologically acceptable plastic such as FDA-approved acrylic plastic and may have the length of 250 to 300 mm with the outer diameter of 20 mm. The entire outer shell 22 may comprise a single rigid unit.

The outer shell 22 is intended for insertion into the patient's rectum and during such an insertion, the distal end of the outer shell should be closed, and this function is fulfilled by an end tip 36 (FIGS. 1 and 2) on the distal end of the outer tube 24. Since in the condition shown in FIG. 2, a gap 32 is formed between the outer surface 36a of the end tip 36 and the inner surface 24a of the outer tube 24, the outer tube 24 is provided on its distal end with an inner shoulder 28 that support the distal end of the end tip 36 against lateral movements. The inner surface 24a may have a reflecting coating (not shown) for the purposes described later. A side handle 38 (FIG. 1) is formed on the side of the outer shell 22 close to its proximal end for insertion of an optical fiber bundle 30 of a lighting device (not shown in the handle).

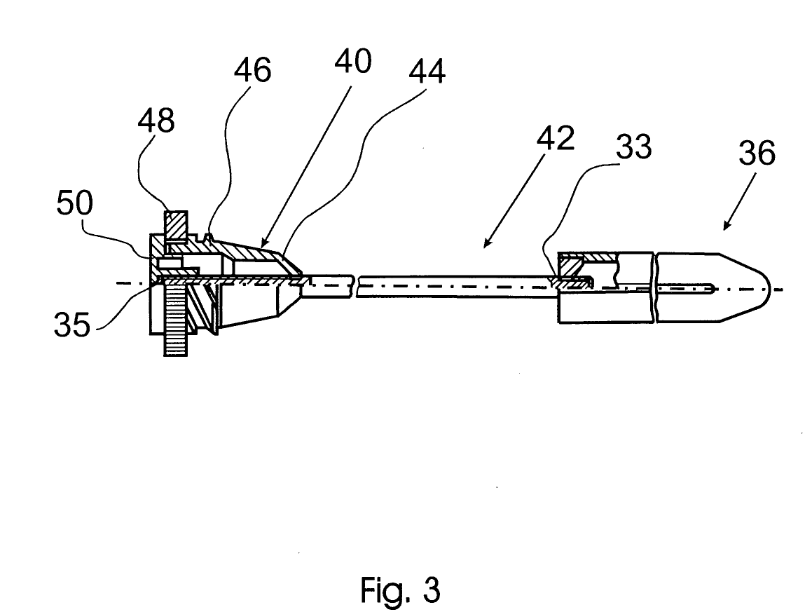

The rear end of the end tip 36 is rigidly connected, e.g., by means of a thread 33, to a stem 34. The rear or proximal end of the stem 34 is attached, e.g., screwed at 35, to a threaded tubular lead screw member 40 shown in more detail in FIG. 3, which is a longitudinal partially sectional view of an end tip assembly 42. More specifically, this assembly that consists of the aforementioned end tip 36 with the stem 34, and the threaded lead screw member 40 can be considered as an integral unit that can be inserted into the outer shell 22 and removed therefrom as a single body.

As can be seen from FIG. 3, the tubular lead screw member 40 has an outer tapered surface 44. The taper may be, e.g., from 5° to 30°. The tubular screw member 40 has a cylindrical outer portion with an outer thread 46 that should provide quick and easy engagement with a mating part (not shown in FIG. 3). The thread 46 may be, e.g., a four-start trapezoidal thread with the thread tooth height preferably satisfying the following condition: h<0.5p, where p is a thread pitch. The thread may have all parameters, except for the tooth height, corresponding to appropriate international or national standards. These parameters are given only as an example.

For convenience of handling of the end tip assembly 42, the tubular screw member 40 has a knurled flange 48 (FIGS. 1 and 3) located behind the thread 46 in the proximal direction. The rear open end of the tubular lead screw member 40 is closed by a cover 50.

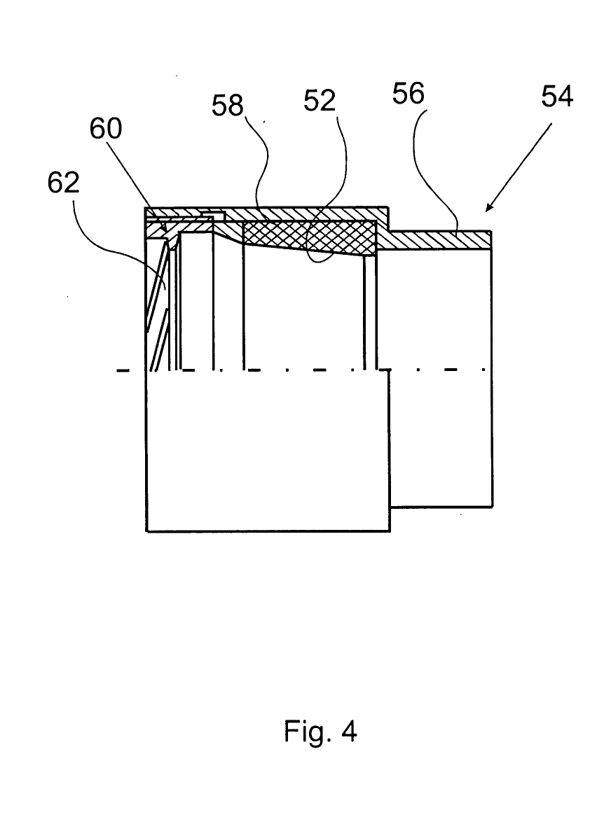

The outer tapered surface 44 of the tubular lead screw member 40 is intended for engagement with a mating inner tapered surface 52 of a lock member 54 shown in FIG. 4. The lock member 54 is an integral part of the outer shell 22 and is inserted into and rigidly secured to the proximal end of the outer shell 22. As shown in FIG. 4, the lock member 54 has a metal casing 56 with a plastic insert 58 and a metal lead nut 60. The insert have an inner tapered surface 52 with substantially the same taper angle as the mating outer tapered surface 44 of the screw member 40. The plastic insert may be molded, e.g., from a fluoroelastomer, or from a similar material which provides good sliding conditions of the inner plastic tapered surface over the outer stainless-steel tapered surface 44 of the screw member 40.

The lead nut 60 has an inner thread 62 with the same number of starts, lead angle, and pitch p as the outer thread 46 of the tubular screw member 40. When the end tip assembly 42 is inserted into the outer shell 22, and the screw member 40 is inserted into the lock member 54, rotation of the lead nut 60 will cause, through engagement between the outer and inner threads 46 and 62, respectively, rigid fixation of the end tip assembly 42 in the outer shell 22. It is recommended that the lead angle of the thread be selected so that tightening of the threads could be achieved by turning the flange 48 by 10 to 20°.

Prior to operation, the end tip assembly 42 consisting of an end tip 36, stem 34, and screw member 40 is inserted as an integral unit into the outer shell 22 through its proximal end into its extreme forward position, in which, as shown in FIGS. 1 and 2, the interior of the outer shell 22 is completely closed by the assembly 42, and end of the tip 36 projects through the distal end of the outer shell 22 and in the form of a smooth rounded end that facilitates subsequent insertion of the endoscope into the patient's rectum. After the assembly 42 is inserted into the outer shell 22, the position of the assembly 42, and thus of the projecting portion of the tip 36, is fixed by engaging the threads 46 and 62. The above-described insertion is facilitated due to engagement of metal-plastic friction pair on tapered surfaces.

With the interior of the outer shell closed by the assembly 42, the outer shell is inserted into the patient's rectum in accordance with the procedure specified by the clinical rules. When the endoscope insertion procedure is completed, the assembly 42 is extracted from the outer shell 22, that remains inside the patient's rectum, by turning the flange 48 (FIGS. 1 and 3) in the direction of unscrewing the threads 46 and 62.

It is understood that extraction of the assembly 42 from the outer shell 22 leaves the interior of the outer shell completely open for insertion of any required tools (not shown) such as biopsy samplers, laser treatment tip, polyp cutters, etc. The zone of operation or observation will be illuminated by light emitted from the distal end of the cylindrical optical fiber bundle 30 (FIG. 2).

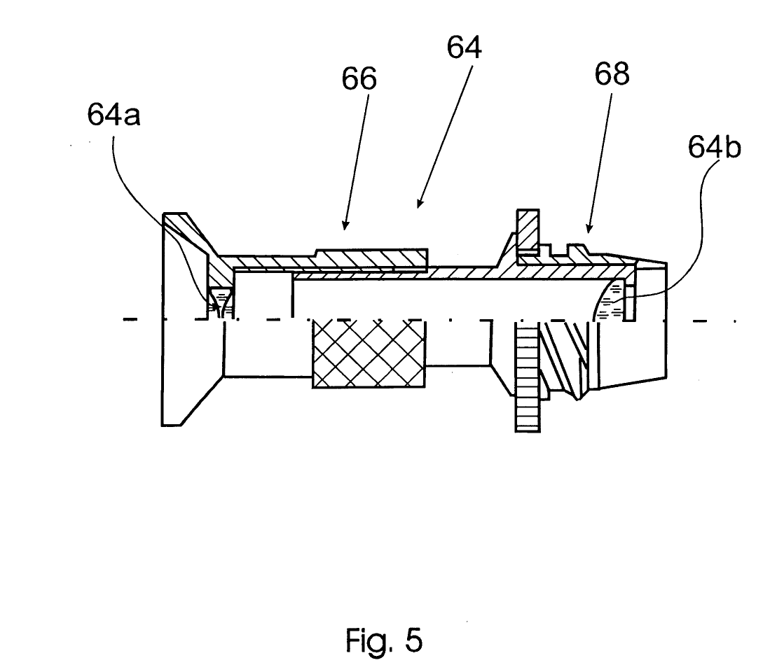

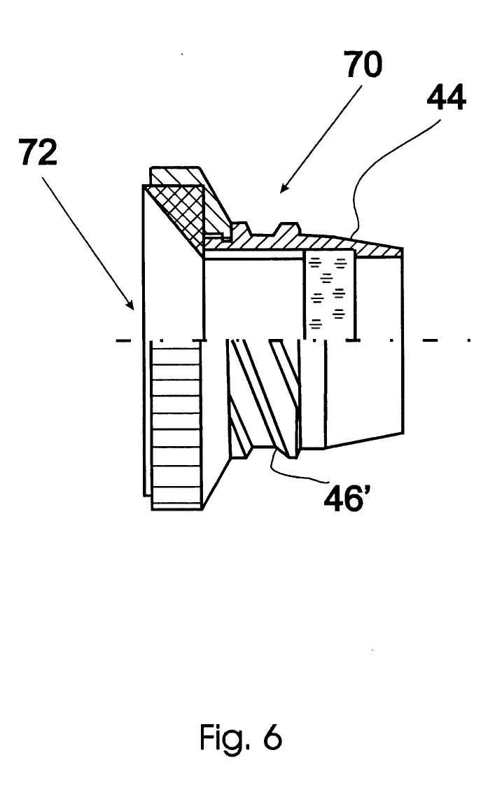

If necessary, prior to operation, the area of interest can be observed with magnification through the use of an optical eyepiece 64, or the like shown in FIG. 5. Such a device may comprise a conventional telescopic optical viewer with two lenses 64a and 64b inserted into a body 66 with possibility of axial movement with respect to each other for optical adjustment. The details of such a device are optional except for the distal end 68 with connection elements that should correspond to the respective receiving connection elements of an adapter screw member 70, which is shown in FIG. 6. This adapter screw member 70 is similar to the screw member 40 in that it is made from a metal, e.g., stainless steel and has the same geometry of the outer thread 46′ and of the outer tapered portion 44′ for engagement with the inner thread 62 and the tapered surface 52, respectively, of the plastic insert 58 of the lock member 54. The difference between both screw members 40 and 70 is that the diameter of the inner opening of the screw member 70 is greater in order not to obscure the field of observation and that the rear or proximal end 72 of the nut 70 has a geometry mating the corresponding connection portion, i.e., the distal end 68, of the optical eyepiece 64.

When the area of the interest is observed with the use of the eyepiece 64, it is illuminated with the light emitted from the end of the optical fiber bundle 30. The illumination conditions are improved by scattering the light from the reflecting surface 24a (FIG. 2).

In fact, any replaceable tool or instrument, such as biopsy samplers, polyp cutters, etc. having the connection parts mating the connecting elements on the proximal ends of the end tip assembly 40 shown in FIG. 3, or the adapter screw member 70, can be inserted into the outer shell 22. Multiple-start trapezoidal threads 46, 46′, and 62 with an appropriate lead angle of the thread that ensures complete lock of the threads with rotation of the screw, e.g., by 10 to 20° provides quick and easy replacement of the tools inserted into and extracted from the outer shell.

Thus, it has been shown that the invention provides a universal endoscope for proctological purposes, which is simple in construction, convenient for use and sterilization, is provided with an optical observation unit located on the distal end of the outer shell in direct proximity to the area of interest, provides quick one-step removal of the observation unit opening access to any tools that could be inserted into the outer shell of the endoscope through the completely open proximal end, has an improved locking device for the replaceable optics with the use of a metal-plastic friction pair that improves sliding, and prevents jamming of connection components.

Although the invention has been shown and described with reference to specific embodiments, it is understood that these embodiments should not be construed as limiting the application of the invention, so that any changes and modifications are possible, provided they do not depart from the scope of the patent claims. For example, the optical viewing device may comprise an elongated telescopic member with the distal-end lens insertable to a position in close proximity to the area of observation. The metal and plastic materials, as well as the angles of tapers and threads mentioned in the description are given only as examples. The endoscope itself is not limited to constructions for proctological use and the principle of locking of the inserts into the outer shells of the endoscope can be used in gastroscopes, laporoscopes, or the like. Although a plastic member was shown as a tapered insert 58 of the locking device 54 and the metal member was shown as the lead screw 40, the principle of using a metal-plastic pair will not be violated if the insert 58 is made of metal and the lead screw 40 is made of plastic.

1. A universal endoscope for insertion into a patient's body lumen comprising: a tubular outer shell having a proximal end, a distal end, and a hollow interior; a locking member rigidly connected to said proximal end of said tubular outer shell; a replaceable end tip assembly for insertion into said tubular outer shell through said proximal end of said tubular outer shell, said replaceable end tip assembly having a proximal end and a distal end and comprising a rounded tip member with a smooth surface on said distal end of said replaceable end tip assembly and a lead screw member on said proximal end of said replaceable end tip assembly; and illumination means; said locking member having an inner tapered member with an inner tapered surface while said lead screw member having an outer tapered member with an outer surface, said inner tapered surface having substantially the same taper angle as said outer tapered surface; said outer taper member and said inner taper member forming a metal-plastic friction pair wherein either one of said outer taper member and said inner taper member is made of metal while the other one is made of plastic.

2. The endoscope of claim 1, wherein said plastic is a fluoroelastomer.

3. The endoscope of claim 2, wherein said metal is a stainless steel.

4. The endoscope of claim 1, wherein said lead screw member has an outer thread, and said locking member having an inner thread engageable with said outer thread so that when said outer thread and said inner thread are tightened, said metal-plastic friction pair is in a tight friction engagement.

5. The endoscope of claim 2 wherein said lead screw member has an outer thread, and said locking member having an inner thread engageable with said outer thread so that when said outer thread and said inner thread are tightened, said metal-plastic friction pair is in a tight friction engagement.

6. The endoscope of claim 3 wherein said lead screw member has an outer thread, and said locking member having an inner thread engageable with said outer thread so that when said outer thread and said inner thread are tightened, said metal-plastic friction pair is in a tight friction engagement.

7. The endoscope of claim 5, wherein outer thread and said inner thread are multiple-start trapezoidal threads satisfying the following condition: h<0.5p, where p is a thread pitch.

8. The endoscope of claim 7, wherein said outer thread and said inner thread have a thread lead angle that provides said tight friction engagement with rotation of said lead screw member by 10 to 20°.

9. The endoscope of claim 6, wherein outer thread and said inner thread are multiple-start trapezoidal threads satisfying the following condition: h<0.5p, where p is a thread pitch.

10. The endoscope of claim 9, wherein said outer thread and said inner thread have a thread lead angle that provides said tight friction engagement with rotation of said lead screw member by 10 to 20°.

11. The endoscope of claim 1, wherein said outer shell comprising an outer tube and an inner sleeve inserted into said outer tube with an annular space formed therebetween, and wherein said illumination means comprises an optical fiber bundle that has a light emitting portion formed into a cylindrical configuration placed into said annular space and fixed betweens said outer tube and inner sleeve, said light emitting portion being located in the area of said distal end of said outer shell.

12. The endoscope of claim 5, wherein said outer shell comprising an outer tube and an inner sleeve inserted into said outer tube with an annular space formed therebetween, and wherein said illumination means comprises an optical fiber bundle that has a light emitting portion formed into a cylindrical configuration placed into said annular space and fixed betweens said outer tube and inner sleeve, said light emitting portion being located in the area of said distal end of said outer shell.

13. The endoscope of claim 6, wherein said outer shell comprising an outer tube and an inner sleeve inserted into said outer tube with an annular space formed therebetween, and wherein said illumination means comprises an optical fiber bundle that has a light emitting portion formed into a cylindrical configuration placed into said annular space and fixed betweens said outer tube and inner sleeve, said light emitting portion being located in the area of said distal end of said outer shell.

14. The endoscope of claim 9, wherein said outer shell comprising an outer tube and an inner sleeve inserted into said outer tube with an annular space formed therebetween, and wherein said illumination means comprises an optical fiber bundle that has a light emitting portion formed into a cylindrical configuration placed into said annular space and fixed betweens said outer tube and inner sleeve, said light emitting portion being located in the area of said distal end of said outer shell.

15. The endoscope of claim 10, wherein said outer shell comprising an outer tube and an inner sleeve inserted into said outer tube with an annular space formed therebetween, and wherein said illumination means comprises an optical fiber bundle that has a light emitting portion formed into a cylindrical configuration placed into said annular space and fixed betweens said outer tube and inner sleeve, said light emitting portion being located in the area of said distal end of said outer shell.

16. The endoscope of claim 5, further comprising an adapter lead screw member that has the same outer thread as said lead screw member on said proximal end of said replaceable end tip assembly, said adapter lead screw member being engages with said inner thread of said locking member when said replaceable end tip assembly is removed from said outer shell, said adapter lead screw having a central opening and means for connecting replaceable tools insertable into said outer shell through said central opening.

17. The endoscope of claim 6, further comprising an adapter lead screw member that has the same outer thread as said lead screw member on said proximal end of said replaceable end tip assembly, said adapter lead screw member being engages with said inner thread of said locking member when said replaceable end tip assembly is removed from said outer shell, said adapter lead screw having a central opening and means for connecting replaceable tools insertable into said outer shell through said central opening.

18. The endoscope of claim 14, further comprising an adapter lead screw member that has the same outer thread as said lead screw member on said proximal end of said replaceable end tip assembly, said adapter lead screw member being engages with said inner thread of said locking member when said replaceable end tip assembly is removed from said outer shell, said adapter lead screw having a central opening and means for connecting replaceable tools insertable into said outer shell through said central opening.

19. The endoscope of claim 15, further comprising an adapter lead screw member that has the same outer thread as said lead screw member on said proximal end of said replaceable end tip assembly, said adapter lead screw member being engages with said inner thread of said locking member when said replaceable end tip assembly is removed from said outer shell, said adapter lead screw having a central opening and means for connecting replaceable tools insertable into said outer shell through said central opening.
